(19) United States
(12) Patent Application Publication
(10) Pub. No.: US 2014/0086800 A1
(43) Pub. Date: Mar. 27, 2014

(54) APPARATUS OF VOLATILIZER FOR PHYTONCIDE AIR

(71) Applicant: Yong Sig YOON, Incheon (KR)

(72) Inventor: Yong Sig YOON, Incheon (KR)

(21) Appl. No.: 13/692,170

(22) Filed: Dec. 3, 2012

(30) Foreign Application Priority Data
Sep. 25, 2012 (KR) 10-2012-0106581

(51) Int. Cl. A61L 9/015

(52) U.S. Cl. CPC A61L 9/015
USPC 422/119

(57) ABSTRACT

Provided therein is an apparatus of a volatilizer for phytoncide air, which is a replaceable and exchangeable cartridge type volatilizer, which has an exchange sensor to sense whether or not Hinoki cypress diluted essential oil exists in a reservoir and to let a user know time to replace or exchange the reservoir, and which has slope plates mounted inside the reservoir to make the Hinoki cypress diluted essential oil, which fills the reservoir, go close to and concentrate on an air inlet. The volatilizer for phytoncide air includes: a reservoir having a charging space which is filled with a Hinoki cypress diluted essential oil; slope plates disposed in the charging space of the reservoir; an air inlet and an air outlet respectively formed at an upper portion of the reservoir; and an exchange sensor disposed at a lower portion of the reservoir and connected with a warning light.

### CROSS REFERENCES

Applicant claims foreign priority under Paris Convention to Korean Patent Application No. 10-2012-0106581, filed Sep. 25, 2012, with the Korean Intellectual Property Office, where the entire contents are incorporated herein by reference.

### BACKGROUND OF THE INVENTION

1. Field of the Invention

The present invention relates to an apparatus of a volatilizer, and more particularly, to an apparatus of a volatilizer useful to the human body, which releases phytoncide generated in the woods to thereby provide antibacterial function, sedation, deodorization, stress reduction, reduction of allergies or skin ailments, and removal of formaldehyde and volatile organic compounds (VOCs).

2. Background Art

In general, modern people live in interior spaces, such as houses, offices, buildings, and underground spaces, for a long time.

So, for the modern people who spend most of their time in the interior spaces, pleasant interior environments are being emphasized as an important element to improve the efficiency of business and to maintain their health.

In the meantime, with the improved standards of living, a demand of more pleasant interior environments is increasing. However, there are several problems in that the contents of bad smells and noxious gases are increasing by breathing and smoking in sealed spaces and in that people feel difficulty in breathing and displeasure due to various kinds of dusts generated from clothes or other things when they live in the interior spaces for a long time.

In order to solve the above-mentioned problems, various kinds of air cleaners for removing dusts, bad smells and noxious gases contained in the inside air have been developed.

Such air cleaners have a structure that it inhales the inside air, removes dusts, bad smells and harmful elements from the inhaled inside air, and releases the purified air to the interior space, and are divided into electric precipitation type air cleaners and filtration type air cleaners.

In the meantime, all trees and plants in woods release phytoncide, which is an aromatic substance, in order to protect themselves from various harmful insects and bacteria.

It is known that phytoncide contains sterilization and insecticide ingredients and terpene-based organic compound, which is the chief ingredient of phytoncide, provides antibacterial function, sedation, deodorization, stress reduction, and so on. Moreover, it has been also reported that if people inhale phytoncide, the concentration of cortisol, which is a stress hormone, is remarkably reduced.

Furthermore, it is also known that phytoncide provides a pleasant feeling through sedation in the central nervous system, extends hours of sleeping, helps people have a good sleep, is good for hyperlipemia, blood clot and cardiac insufficiency by improving the blood circulation system, and serves a strong antibacterial function not to acquire resistance of the human body.

In addition, it is known that phytoncide which is a natural material has no side effects and is highly effective in preventing allergies because it provides an effect that house dust mites avoid it.

As described above, it is well known that phytoncide provides psychological stability, strengthens peripheral blood vessels and cardio pulmonary function, helps treat bronchial asthma and tuberculosis, helpful for strengthening the heart, and sterilizes the skin.

Such phytoncide is generated from Hinoki cypress in large quantities.

In general, Hinoki cypress is a kind of temperate zone evergreen needle-leaf trees, and the home of Hinoki cypress is Japan. In Japan, Hinoki cypresses are mainly growing in Kiso District of Nagano Prefecture, Kochi Prefecture of Shikoku Island, Miyazaki Prefecture of Kyushu Island, and so on.

Hinoki Cypress contains a lot of phytoncide, has an excellent sustaining power and strong water resistance and endogeny, and has its unique wood flavor. Mature Hinoki cypress is about 30 m in height and is about 20 cm in diameter.

Meanwhile, in Korea, Hinoki cypresses are mainly growing in jangseong, jangheung, and Haenam districts of Jeollanamdo. As a result that Korea Forest Research Institute has collected and investigated air of the Hinoki cypress forest at Mt. Chukryeong, it has been revealed that extracts of Hinoki cypress leaves contain sabinene, which removes molds causing asthma, 38 times more than pine needle extract and Korean Hinoki cypresses have characteristics similar to Japanese Hinoki cypresses, which are superior in quality.

### SUMMARY OF THE INVENTION

Accordingly, the present invention has been made to solve the above-mentioned problems occurring in the prior arts, and it is an object of the present invention to provide an apparatus of a volatilizer for phytoncide air, which is a replaceable and exchangeable cartridge type volatilizer, which has an exchange sensor to sense whether or not Hinoki cypress diluted essential oil exists in a reservoir and to let a user know time to replace or exchange the reservoir, and which has slope plates mounted inside the reservoir to make the Hinoki cypress diluted essential oil, which fills the reservoir, go close to and concentrate on an air inlet.

It is another object of the present invention to provide an apparatus of a volatilizer useful to the human body, which easily releases the Hinoki cypress diluted essential oil generating phytoncide, which keeps the uniform concentration during the use, and which provides antibacterial function, sedation, deodorization, stress reduction, reduction of allergies or skin ailments, and an easy removal of formaldehyde and volatile organic compounds (VOCs).

To achieve the above objects, the present invention provides an apparatus of a volatilizer for phytoncide air including: a reservoir having a charging space which is filled with a Hinoki cypress diluted essential oil; slope plates disposed in the charging space of the reservoir; an air inlet and an air outlet respectively formed at an upper portion of the reservoir; and an exchange sensor disposed at a lower portion of the reservoir and connected with a warning light.

Here, at least one of the slope plates is mounted inside the charging space of the reservoir and is inclined at a predetermined angle toward a lower surface from an inner face of the charging space.

Moreover, the exchange sensor comprises at least two sensor pipes, and turns on the warning light when at least one of the sensor pipes is exposed to the air.

In the meantime, the Hinoki cypress diluted essential oil contains 1.46 to 1.97% by weight of concentrated Hinoki cypress essential oil, 68.23 to 77.52% by weight of alkaline purified water, 19.38 to 29.24% by weight of Hinoki cypress water, and 1.07 to 1.16% by weight of SAP (Super Absorbent Polymer).

As described above, the apparatus of the volatilizer for phytoncide air according to the present invention easily keeps the uniform concentration of the Hinoki cypress diluted essential oil, easily maintains phytoncide for a certain period of time, has no side effects and is highly effective in preventing allergies because it provides an effect that house dust mites avoid it, serves a strong antibacterial function not to acquire resistance of the human body due to eco-friendly and natural phytoncide, and is harmless to humans.

Moreover, the apparatus of the volatilizer for phytoncide air according to the present invention releases phytoncide having efficiencies useful to the human body, because it provides antibacterial function, sedation, deodorization, stress reduction, reduction of allergies or skin ailments, and an easy removal of formaldehyde and volatile organic compounds (VOCs). Furthermore, if people inhale phytoncide, it remarkably reduces the concentration of cortisol, which is a stress hormone, provides a psychological stability and a pleasant feeling through sedation in the central nervous system, extends hours of sleeping, helps people have a good sleep, is good for hyperlipemia, blood clot and cardiac insufficiency and strengthens the cardio pulmonary function by improving the blood circulation system such as peripheral blood vessels, and sterilizes the skin.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a schematic diagram of an apparatus of a volatilizer for phytoncide air according to the present invention; and

FIG. 2 is a perspective view, in brief, of a reservoir of the volatilizer for phytoncide air according to the present invention.

### DETAILED DESCRIPTION OF THE PREFERRED EMBODIMENT

Reference will be now made in detail to the preferred embodiment of the present invention with reference to the attached drawings. It would be understood that the following embodiment of the present invention does not restrict the scope of claims of the present invention and is just used to exemplify. Moreover, it will be understood by those of ordinary skill in the art that various changes in form and details may be made therein without changing the technical idea or essential characteristics of the present invention as defined by the following claims.

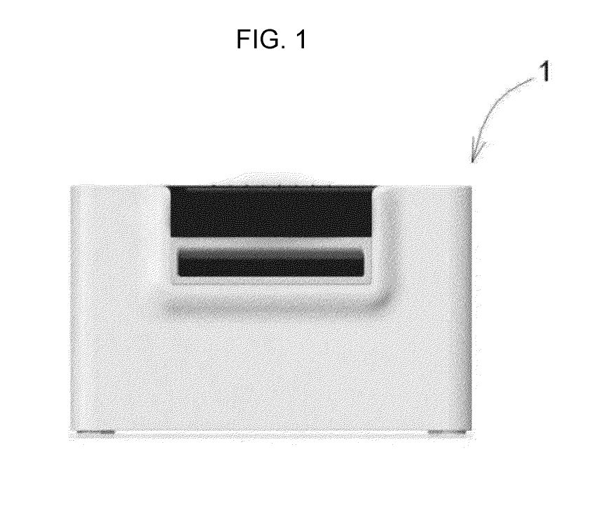

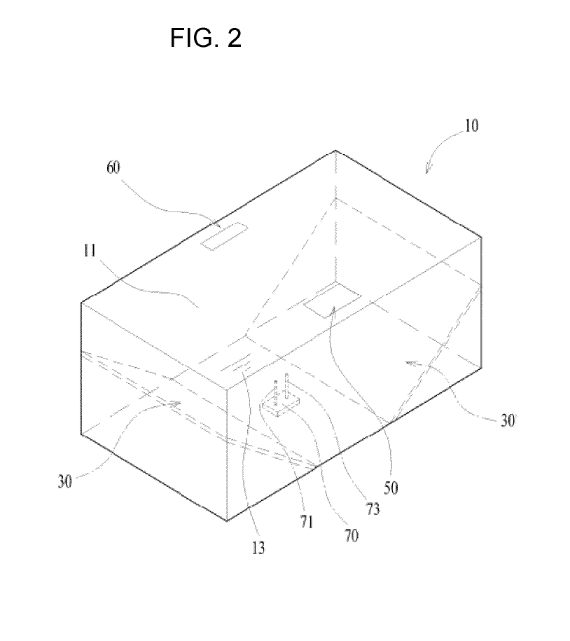

FIG. 1 is a schematic diagram of an apparatus of a volatilizer for phytoncide air according to the present invention, and FIG. 2 is a perspective view, in brief, of a reservoir of the volatilizer for phytoncide air according to the present invention.

As shown in the drawings, the volatilizer 1 according to the present invention includes a reservoir 10, slope plates 30 and 30′, an air inlet 50, an air outlet 60, and an exchange sensor 70.

The reservoir 10 is in an approximately hexahedral shape, has a charging space 11 that Hinoki cypress diluted essential oil 13 fills, and is in a replaceable and exchangeable cartridge type.

Here, the reservoir 10 includes a reservoir body (not shown) and a reservoir cover (not shown) removably joined to the upper portion of the reservoir body. Between the reservoir body and the reservoir cover, a silicon packing which is harmless to the human body and has good chemical resistance is mounted, or EVA (Ethylene Vinylacetate) is adhered to prevent separation of the reservoir cover and to secure the safety according to consumer needs.

In this instance, in order to check sealability after the reservoir body and the reservoir cover of the reservoir 10 are assembled and joined together, the reservoir body and the reservoir cover are joined with each other by means of a lock or by means of a lock and an adhesion method, but the assembling and joining method is not restricted to the above.

Furthermore, it is preferable that the reservoir 10 is made of a random polypropylene copolymer material out of olefin-based materials in consideration of chemical resistance, semitransparentness, and impact resistance to provide stability, but the present invention is not restricted to the above.

The slope plates 30 and 30′ are disposed inside the charging space 11 of the reservoir 10. In this instance, at least one of the slope plates 30 and 30′ is provided inside the charging space 11 of the reservoir 10 and is inclined at a predetermined angle toward a lower surface of the inner face of the charging space 11.

In other words, the slope plates 30 and 30′ are respectively disposed at both end portions of the charging spaces 11 in a longitudinal direction in order to concentrate and transfer the Hinoki cypress diluted essential oil 13, which is consumed according to the duration of oil usage and the amount of oil used, onto lower portions of the air inlet 50 and the air outlet 60. Additionally, the slope plates 30 and 30′ are respectively inclined in such a fashion that one side is in contact with the inner face of the charging space 11 and the other side is in contact with the lower face of the charging space 11.

In this embodiment of the present invention, the slope plates 30 and 30′ are respectively inclined in such a fashion that they are in contact with the inner face and the lower face of the charging space 11 of the reservoir 10 in the longitudinal direction, but it is also possible that the slope plates 30 and 30′ are respectively inclined in such a fashion that they are in contact with the inner face and the lower face of the charging space 11 of the reservoir 10 in a width direction, and the number of the slope plates 30 and 30′ are not restricted to the above.

Moreover, the slope plates 30 and 30′ are respectively inclined at the predetermined angle in such a fashion that they are in contact with the central portion of the inner face and a side of the lower face of the charging space 11, but the inclination angles and the mounted positions of the slope plates 30 and 30′ are not restricted to the above.

The air inlet 50 and the air outlet 60 are respectively formed at the upper portion of the reservoir 10. That is, the air inlet 50 and the air outlet 60 respectively penetrate the central portion of the upper portion of the reservoir 10.

In this instance, in order to discharge the air introduced into the air inlet 50 through the air outlet 60, it is preferable that the air inlet 50 and the air outlet 60 are positioned in the upper portion of the reservoir 10 at right angles to each other.

The exchange sensor 70 is disposed at the lower portion of the reservoir 10, and is connected to a warning light (not shown) exposed to the outside of the volatilizer 1.

Here, the exchange sensor 70 includes two sensor pipes 71 and 73 having electrical resistance, and if one of the sensor pipes 71 and 73 is exposed to the air, the exchange sensor 70 senses it and turns on the warning light.

In other words, the exchange sensor 70 senses whether or not Hinoki cypress diluted essential oil exists in the charging space 11 of the reservoir 10 in order to let a user know the time to exchange and replace the reservoir 10. Accordingly, when one of the sensor pipes 71 and 73 of the exchange sensor 70 is exposed to the air, the exchange sensor 70 turns on the warning light of the volatilizer 1, so that the user can exchange or replace the reservoir 10 of the cartridge type.

For this, the sensor pipes 71 and 73 of the exchange sensor 70 are respectively embedded in such a way as to be partially exposed toward the lower surface of the charging space 11 of the reservoir 10.

As described above, the exchange sensor 70 mounted on the lower portion of the charging space 11 of the reservoir 10 detects a resistance value of 300 kΩ to 400 kΩ when the charging space 11 is filled with the Hinoki cypress diluted essential oil 13 but detects a value of air resistance of ∞ when the Hinoki cypress diluted essential oil 13 is exhausted in the charging space 11, so that the user can realize the time to replace or exchange the cartridge type reservoir 10.

Here, it is preferable that the warning light is a red LED lamp, and when the red LED lamp is turned on, the user replaces or exchanges the cartridge type reservoir 10.

In the meantime, the Hinoki cypress diluted essential oil filling the charging space 11 of the reservoir 10 contains 1.46 to 1.97% by weight of concentrated Hinoki cypress essential oil, 68.23 to 77.52% by weight of alkaline purified water, 19.38 to 29.24% by weight of Hinoki cypress water, and 1.07 to 1.16% by weight of SAP (Super Absorbent Polymer).

Hereinafter, an operational process of the apparatus of the volatilizer 1 according to the present invention will be described.

First, the air supplied into the volatilizer 1 by wind generated from a blower is introduced into the air inlet 50. That is, the blower mounted on the volatilizer 1 blows the outside air into the volatilizer 1, and then, the air is introduced into the air inlet formed in the upper portion of the reservoir 10.

Because a fan of the blower mounted on the volatilizer 1 blows the outside air into the reservoir 10, the volatilizer 1 can keep the uniform concentration while the Hinoki cypress diluted essential oil 13 filling the charging space 11 of the reservoir 10 is used, such that phytoncide can be kept in the charging space 11 for a certain period of time.

For this, it is preferable that the blower is positioned and mounted at the upper portion of the air inlet 50 perforated in the upper portion of the reservoir 10 to thereby introduce the outside air into the charging space 11.

As described above, the outside air introduced into the air inlet 50 collides with the Hinoki cypress diluted essential oil 13 filling the reservoir 10 and is volatilized, and the volatilized air containing the Hinoki cypress diluted essential oil 13 is diffused into the air while being discharged through the air outlet 60 formed in the upper portion of the reservoir 10.

The air introduced from the outside through the air inlet 50 by the blower is mixed with the Hinoki cypress diluted essential oil 13, and then, is discharged to the outside through the air outlet 60, such that the volatilizer 1 installed in a room discharges and releases phytoncide into the room to purify the indoor air.

Here, phytoncide generated from the volatilizer 1 and discharged and released into the room is almost identical with phytoncide released from woods, and hence, the volatilizer 1 according to the present invention can provide antibacterial function, sedation, deodorization, stress reduction, reduction of allergies or skin ailments, and removal of formaldehyde and volatile organic compounds (VOCs), and contribute to human's health and lives by grafting the useful effects of woods onto the city life using phytoncide.

In this instance, when the blower is operated to discharge the air volatilized in the Hinoki cypress diluted essential oil 13 through the volatilizer 1, the volatilizer 1 may be operated in a sleep mode, a regular mode and a strong mode according to the discharge period of time.

In other words, when the blower is operated to introduce the outside air into the charging space 11 of the reservoir 10 of the volatilizer 1 and discharge the air volatilized in the Hinoki cypress diluted essential oil 13, the volatilizer 1 may be operated in the sleep mode that it is operated for one minute and stops for nine minutes, in the regular mode that it is operated for one minute and stops for four minutes, or in the strong mode that it is operated for one minute and stops for two minutes, so that the volatilizer 1 can control its operation period of time and wind strength according to the user's needs.

In this embodiment, the volatilizer 1 can control its operation period of time and wind strength in three stages, namely, the sleep mode, the regular mode, and the strong mode, but is not restricted to the above.

Meanwhile, when the Hinoki cypress diluted essential oil 13 filling the charging space 11 of the reservoir 10 is exhausted, the exchange sensor mounted at the lower portion of the charging space 11 senses it to let the user realize.

That is, when the Hinoki cypress diluted essential oil 13 fully fills the charging space 11 of the reservoir 10, the sensor pipes 71 and 73 of the exchange sensor 70 exposedly mounted on the lower portion of the charging space 11 detects the resistance value of 300 kΩ to 400 kΩ, but, when the Hinoki cypress diluted essential oil 13 is exhausted in the charging space 11 of the reservoir 10, the sensor pipes 71 and 73 of the exchange sensor 70 exposedly mounted on the lower portion of the charging space 11 detects the resistance value of ∞, which is the value of air resistance, so that the user can realize the time to replace or exchange the reservoir 10 through the exchange sensor 70.

In this instance, it is preferable that the exchange sensor 70 is connected with the warning light mounted outside the volatilizer 1 to thereby let the user realize the time to replace and exchange the cartridge type reservoir 10.

As described above, the reservoir 10 of the volatilizer 1 according to the present invention adopts the cartridge type exchange method in order to provide the user with convenience in use, so that the user can replace or exchange the reservoir 10 by opening the cover of the volatilizer 1.

Meanwhile, how to manufacture the Hinoki cypress diluted essential oil 13 by distilling Hinoki cypress will be described. Because Hinoki cypress has characteristics that the aroma of Hinoki cypress leaves or trunks is mild when the Hinoki cypress leaves or trunks are distilled within an hour after being collected but becomes stronger after being dried, the Hinoki cypress diluted essential oil 13 is manufactured and produced by differing the drying method and the drying period of time according to uses.

Furthermore, because the Hinoki cypress diluted essential oil 13 is produced using the vapor distillation method, the Hinoki cypress diluted essential oil 13 is produced in a low yield of about 1%. During the distillation process, Hinoki cypress produces Hinoki cypress oil and Hinoki cypress water as a by-product, and the two materials become complete products through the refinement process.

Here, the Hinoki cypress oil has a specific gravity of 0.89 because it contains volatile materials, but the Hinoki cypress water has a specific gravity of 0.99, which is similar to water.

The Hinoki cypress oil manufactured through the above is produced through the vapor distillation process, and properties of the Hinoki cypress oil and the Hinoki cypress water produced through the vapor distillation process are indicated in the following Table 1.

TABLE 1

| | Phase | | Reflective | Specific |
|---|---|---|---|---|
| P/name | state | PH | index | gravity |
| Hinoki | Liquid | 3.7~3.8 | 1.48 | 0.899 |
| essential oil | | | | |
| Hinoki water | liquid | 3.8~3.9 | 1.33 | 0.99 |

In the meantime, because general Hinoki cypress contains oil, Hinoki cypress oil is used by being properly mixed with water according to the usage. Moreover, because water and oil are low in mutual miscibility and activity resulting from their molecular structures, water and oil are combined chemically and physically using emulsion of water by mixing a surfactant.

In this instance, the surfactant has the oleophilic group, which is a functional group combinable with the Hinoki cypress oil, and the hydrophilic group, which is a functional group combinable with water, and has characteristics of a compound to stabilize water and oil during a predetermined period of time or at predetermined temperature.

However, the present invention uses SAP (Super Absorbent Polymer) in order to substitute for the surfactant applied for emulsion of water and oil. The Hinoki cypress diluted essential oil 13 is harmless to the human body because it is eco-friendly and is obtained from the nature, and the SAP (Super Absorbent Polymer) is applied to the Hinoki cypress oil and water in order to maximize the phytoncide effect of Hinoki cypress.

The SAP applied in lieu of the surfactant can absorb water and oil uniformly by a physical dispersion.

For this, because the Hinoki cypress oil and water are dispersed physically and the diluted solution uniformly dispersed to the SAP must be absorbed as soon as possible before the uniformly dispersed oil is coagulated, the SAP does not have any influence on volatility of highly volatile Hinoki cypress oil.

### Experiment Example

Hinoki cypress oil is heated within a range of 25° C. to 30° C. and stirred for about two minutes using a stirrer with a proper area at speed of 150 rpm to disperse the Hinoki cypress oil and water physically and uniformly.

Moreover, the SAP (Super Absorbent Polymer) is added to the physically dispersed solution, and the solution is stirred at uniform speed for one to two minutes in order to prevent self-agglomeration of the Hinoki cypress oil. When a predetermined amount of the solution is absorbed, the solution is shaken in such a fashion that the remaining solution is absorbed to the SAP as much as possible.

In the case of the remaining solution, an amount of the Hinoki cypress oil may be more than that of water, and it may cause a difference in concentration when the solution is volatilized, and hence, it may differentiate the initial perceptual quality from the perceptual quality after a predetermined period of time passes.

### Embodiment 1

Concentrated Hinoki cypress essential oil: 1.46% by weight
Alkaline purified water: 68.23% by weight
Hinoki cypress water: 29.24% by weight
SAP (Super Absorbent Polymer): 1.07% by weight

### Embodiment 2

Concentrated Hinoki cypress essential oil: 1.94% by weight
Alkaline purified water: 77.52% by weight
Hinoki cypress water: 19.38% by weight
SAP (Super Absorbent Polymer): 1.16% by weight

### Compared Example

Hinoki cypress essential oil: 1% to 5% by weight
Solubilizer: 2% to 10% by weight
Alcohole: 5% to 15% by weight
Purifier water: 70% to 85% by weight

Conventional product use the oil including the Hinoki cypress essential oil and pine tree oil or nut pine oil for reduction of cost.

Conventional product use the solubilizer double the amount of the Hinoki cypress essential oil, and in the case of synthesized solubilizer, it is about 10% to 20% of the Hinoki cypress essential oil.

TABLE 2

| | Generation of | Antibacterial | |
|---|---|---|---|
| | phytoncide | action | deodorization |
| Embodiment 1 | Excellent | Excellent | Excellent |
| Embodiment 2 | Excellent | Excellent | Excellent |
| Compared | Normal | Normal | Normal |
| example | | | |

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An apparatus of a volatilizer for phytoncide air comprising:
a reservoir having a charging space which is filled with a Hinoki cypress diluted essential oil;
slope plates disposed in the charging space of the reservoir;
an air inlet and an air outlet respectively formed at an upper portion of the reservoir; and
an exchange sensor disposed at a lower portion of the reservoir and connected with a warning light.

2. The apparatus of the volatilizer according to claim 1, wherein at least one of the slope plates is mounted inside the charging space of the reservoir and is inclined at a predetermined angle toward a lower surface from an inner face of the charging space.

3. The apparatus of the volatilizer according to claim 1, wherein the exchange sensor comprises at least two sensor pipes, and turns on the warning light when at least one of the sensor pipes is exposed to the air.

4. The apparatus of the volatilizer according to claim 1, wherein the Hinoki cypress diluted essential oil contains 1.46 to 1.97% by weight of concentrated Hinoki cypress essential oil, 68.23 to 77.52% by weight of alkaline purified water, 19.38 to 29.24% by weight of Hinoki cypress water, and 1.07 to 1.16% by weight of SAP (Super Absorbent Polymer).
